(19) United States
(12) Patent Application Publication
(10) Pub. No.: US 2021/0260199 A1
(43) Pub. Date: Aug. 26, 2021

(54) NOVEL NARINGENIN/LIPOIC ACID CONJUGATE COMPOUND AND USE THEREOF

(71) Applicant: Yang-Heon SONG, Daejeon (KR)

(72) Inventors: Yang-Heon SONG, Daejeon (KR); Tarek SALEH, Guelph, Ontario (CA)

(21) Appl. No.: 17/253,250

(22) PCT Filed: Jun. 19, 2019
(86) PCT No.: PCT/KR2019/007369

§ 371 Date: Dec. 17, 2020

(30) Foreign Application Priority Data
Jun. 19, 2018 (KR) 10-2018-0070315

(51) Int. Cl. A61K 47/55; A61K 8/49; A61Q 19/02; A61Q 17/00; A61Q 17/04; A61Q 19/08; A61P 9/10; A61P 25/16; A61P 25/28; A61K 31/385; A61K 31/353; A61K 9/00; A23L 33/10; A23L 33/00

(52) U.S. Cl. CPC A61K 47/55; A23V 2002/00; A61Q 19/02; A61Q 17/00; A61Q 17/04; A61Q 19/08; A61P 9/10; A61P 25/16; A61P 25/28; A61K 31/385; A61K 31/353; A61K 9/0056; A23L 33/10; A23L 33/40; A61K 8/4986

(57) ABSTRACT

The present invention relates to a novel compound conjugated with naringenin and lipoic acid which are linked with a linker, and a use thereof.

### TECHNICAL FIELD

The present invention relates to a novel compound conjugated with naringenin and lipoic acid which are linked with a linker, and a use thereof.

### BACKGROUND ART

Naringenin, a type of flavonoid, is a substance mainly contained in citrus fruits and is one of major antioxidants and exhibits various physiological activities. It has been known that the naringenin has antioxidation, anti-inflammatory, anti-tumor, and anti-cancer effects through many animal and human experiments, and particularly, has an inhibitory effect on the proliferation of rectal cancer or squamous cell carcinoma.

In addition, the naringenin inhibits the generation of active oxygen, increases a mitochondria membrane potential, and inactivates caspase 3 and 7 together to prevent DNA damage.

Therefore, the naringenin has an antioxidant effect of effectively blocking oxidative stress, and anti-inflammatory and anti-cancer effects to prevent and treat associated diseases thereof. Mainly, the naringenin may act as therapeutic agents for cerebral nerve diseases such as stroke (cerebral infarction, cerebral hemorrhage), Parkinson's disease, Alzheimer's disease, and cerebral nerve injury, heart diseases such as myocardial infarction, cataract, rheumatism, diabetes, cancer, etc.

In particular, research is being focused as the naringenin is known to be effective in preventing and treating the stroke in animal experiments. The stroke is one of the leading causes of death and disability in humans. When ischemic cerebral infarction, one of the strokes, occurs once, a specific part of the cerebrum is blocked and oxygen and glucose are not sufficiently supplied to generate active oxygen. While glutamate is released and excessive calcium ions are introduced, a mitochondrial function is lost and immune response and metabolic system are broken, resulting in apoptosis of the cerebral nerve, destruction of the BBB, and brain swelling. As a result, death or a significant physical aftereffect is left behind. Once the onset of cerebral infarction, only the treatment allowed to date is thrombolysis (r-tPA), but only a small fraction of patients to be applied is less than 10%. Moreover, even if a patient undergoes reperfusion, in which blood flows back into the clogged cerebrovascular vessels through thrombolysis, a large amount of active oxygen is generated by the sudden inflow of oxygen. Eventually, casphase-3 is activated to cause apoptosis due to reperfusion, eventually resulting in a large physical impairment by destruction of brain cells in a cerebral infarction site. Therefore, the development of effective and appropriate preventive and therapeutic agents for cerebral infarction is very much required around the world.

Cerebral infarction proceeds through a very complex mechanism, which involves inflammation, excitotoxicity, apoptosis, oxidative stress, and ionic imbalance. Particularly, in the process of inflammatory reaction, NF-kB is involved to regulate various inflammatory genes and products thereof, such as i-NOS, COX-2, TNF-α, IL-1b, IL-6, etc. However, the naringenin inhibits encephalopathy controlled by NF-kB and is involved in an Nrf2 signaling system to reduce oxidative stress or apoptosis, thereby exhibiting an effect of reducing or preventing the damage of cerebral infarction. Thus, the naringenin is a promising candidate substance for the prevention and treatment of cerebral infarction.

In addition, the naringenin has functions of suppressing the breakdown of collagen and promoting collagen production along with whitening of the skin, and also prevents production of melanin and decomposition of elastin, thereby maintaining healthy skin elasticity. Therefore, the naringenin can be used as an external preparation for skin, such as cosmetics.

On the other hand, the lipoic acid is well known as a powerful antioxidant consisting of 8 carbons and 2 sulfurs. Since a C8 position is chiral, R and S types are possible, and only the R type naturally exists. A racemic mixture in which S type lipoic acid and R and S types are mixed may be made by enzymatic and chemical methods. The lipoic acid is both water-soluble and fat-soluble to be dissolved and act in various organs and blood of the human body. The lipoic acid serves as a coenzyme in at least five enzyme reactions. In particular, the lipoic acid may pass through a blood-brain barrier (BBB) to affect a brain function in the brain. The notable role of the lipoic acid has a regenerative capacity by helping glutathione or vitamins C and E as another antioxidant, in addition to the antioxidant capacity of lipoic acid. In addition, dihydrolipoic acid, which is a reduced type with strong activity, also removes active oxygen, and this reduced-type lipoic acid regenerates several antioxidants again to play a role of helping various antioxidant functions, immune functions, and liver functions.

Therefore, the lipoic acid may act very effectively as a strong antioxidant in diseases associated with oxidative stress caused by cellular toxic substances, such as cerebral infarction. As a treatment for cerebral infarction, it is possible to effectively remove a large amount of active oxygen caused by sudden inflow of oxygen in the case of reperfusion. Moreover, the lipoic acid also has a function of preventing the reduction of acetylcholine, which is commonly shown in brain diseases. That is, since the lipoic acid acts as an inhibitor of acetylcholine esterase, the lipoic acid has an effect of increasing the action of acetylcholine at a cerebral nerve junction site of the cerebrum to prevent and treat cognitive function and brain diseases.

In addition, the lipoic acid exhibits a whitening effect on the skin with a strong antioxidant capacity, and has a function of preventing wrinkles and preventing skin aging by participating in the crosslinking of collagen. In particular, tissues and cells promote aging due to free radicals such as active oxygen in the human body or external ultraviolet (photoaging) to alleviate associated inflammatory reactions and maintain or regenerate healthy cells with a capacity of removing free radicals. Therefore, in order to increase the effects of the lipoic acid, various derivatives having low toxicity, easy skin absorption, and excellent stability and solubility have been developed.

However, there has been no study or report on a novel derivative compound derived from naringenin and lipoic acid as a compound which exhibits a synergistic effect of more powerful biological efficacy than useful efficacy of individual naringenin and lipoic acid.

### PRIOR ARTS

[Patent Document]

(Patent Document 1) Korean Patent Registration No. 10-1063876

### DISCLOSURE

### Technical Problem

Accordingly, the present inventors completed the present invention by preparing a novel compound conjugated with naringenin and lipoic acid having useful physiological activities, which are linked with a linker.

Therefore, an object of the present invention is to provide a novel naringenin/lipoic acid conjugate compound and uses as a pharmaceutical composition, a functional health food, and a skin external preparation composition thereof.

### Technical Solution

In order to achieve the above object, the present invention provides a compound represented by the following Chemical Formula 1:

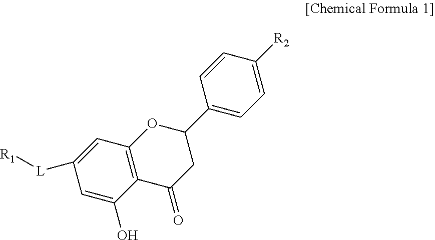

Wherein,

L is a direct bond, or a linker selected from —OCH₂CH₂—, —OCH₂CH₂OCH₂CH₂—, —OCH₂CH₂OCH₂CH₂OCH₂CH₂—, —OCH₂CH₂OCH₂CH₂OCH₂CH₂OCH₂CH₂—, —OCH₂CH₂OCH₂CH₂OCH₂CH₂OCH₂CH₂OCH₂CH₂— and OCH₂CH₂OCH₂CH₂OCH₂CH₂OCH₂CH₂OCH₂CH₂OCH₂CH₂—, and

R₁ and R₂ are each independently OH or lipoic acid represented by the following Chemical Formula 2:

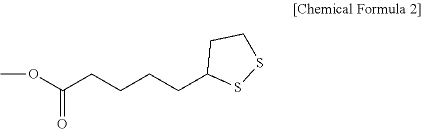

In addition, the present invention provides a pharmaceutical composition for treatment or prevention of stroke, cerebral infarction, cerebral hemorrhage, cognitive impairment, Parkinson's disease, Alzheimer's disease, vascular dementia, memory loss, cerebral nerve injury, etc., comprising the compound according to the present invention or a pharmaceutical salt thereof as an active ingredient.

In addition, the present invention provides a functional health food for improvement or prevention of stroke, cerebral infarction, cerebral hemorrhage, cognitive impairment, Parkinson's disease, Alzheimer's disease, vascular dementia, memory loss, cerebral nerve injury, etc., comprising the compound according to the present invention or a pharmaceutical salt thereof as an active ingredient.

Further, the present invention provides a skin external preparation composition comprising the compound according to the present invention or a pharmaceutical salt thereof as an active ingredient.

Further, the present invention provides a method for preparing the compound according to the present invention by reacting naringenin and lipoic acid.

### Advantageous Effects

According to the present invention, it is possible to provide a compound which exhibits a synergistic effect of naringenin and lipoic acid by linking the naringenin and the lipoic acid having useful physiological activities with a linker to prepare a novel compound. The novel compounds according to the present invention can be used as a pharmaceutical composition or a functional health food useful for the prevention or treatment of cerebral nerve diseases, such as stroke such as cerebral infarction or cerebral hemorrhage, Parkinson's disease, Alzheimer's disease, cognitive disorders, memory loss, and cerebral nerve injury. The novel compounds can be used as cosmetics or a skin external preparation composition having a strong anti-aging effect, such as antioxidation of the skin, sun protection, anti-wrinkling, whitening, anti-inflammatory, production promotion and decomposition prevention of collagen/elastin, and removal of active oxygen.

### DESCRIPTION OF DRAWINGS

FIG. 1 is a graph showing an effect of protecting brain cells by novel compounds of the present invention in an embodiment of the present invention.

FIG. 2 is a graph showing results of evaluating an antioxidant capacity of the novel compounds of the present invention in an embodiment of the present invention.

FIG. 3 shows an effect of the novel compounds of the present invention on a cerebral infarction site in a cerebral infarction animal model according to an embodiment of the present invention.

### MODES OF THE INVENTION

However, hereinafter, embodiments of the present invention will be described in detail with reference to the accompanying drawings. In the following description, detailed descriptions of techniques well-known to those skilled in the art may be omitted. Further, in describing the present invention, the detailed description of associated known functions or constitutions will be omitted if it is determined that they unnecessarily make the gist of the present invention unclear. Terminologies used herein are a terminologies used to properly express embodiments of the present invention, which may vary according to a user, an operator's intention, or customs in the art to which the present invention pertains.

Accordingly, definitions of the terminologies need to be described based on contents throughout this specification. Throughout the specification, unless explicitly described to the contrary, the word “comprise” and variations such as “comprises” or “comprising” will be understood to imply the inclusion of stated elements but not the exclusion of any other elements.

The present invention provides a compound represented by the following Chemical Formula 1:

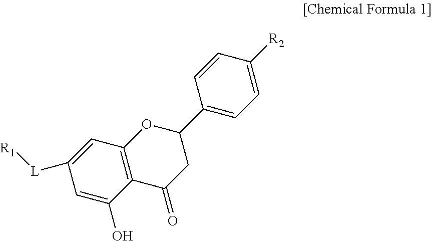

Wherein,

L is a direct bond, or a linker selected from —OCH₂CH₂—, —OCH₂CH₂OCH₂CH₂—, —OCH₂CH₂OCH₂CH₂OCH₂CH₂—, —OCH₂CH₂OCH₂CH₂OCH₂CH₂OCH₂CH₂—, —OCH₂CH₂OCH₂CH₂OCH₂CH₂OCH₂CH₂OCH₂CH₂— and OCH₂CH₂OCH₂CH₂OCH₂CH₂OCH₂CH₂OCH₂CH₂OCH₂CH₂—, and

R₁ and R₂ are each independently OH or lipoic acid represented by the following Chemical Formula 2:

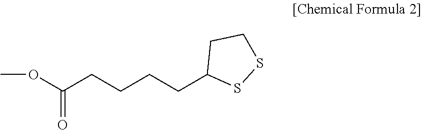

In an embodiment of the present invention, the compound of Chemical Formula 1 above may be prepared by chemically covalently bonding lipoic acid and naringenin via a linker. Specifically, several novel compounds having different binding positions may be prepared by selectively reacting one of hydroxyl groups of naringenin reacting with lipoic acid, and in particular, various compound structures may be prepared through a multi-step reaction using an ether linker.

In an embodiment of the present invention, the compound of Chemical Formula 1 above may include the following compounds:

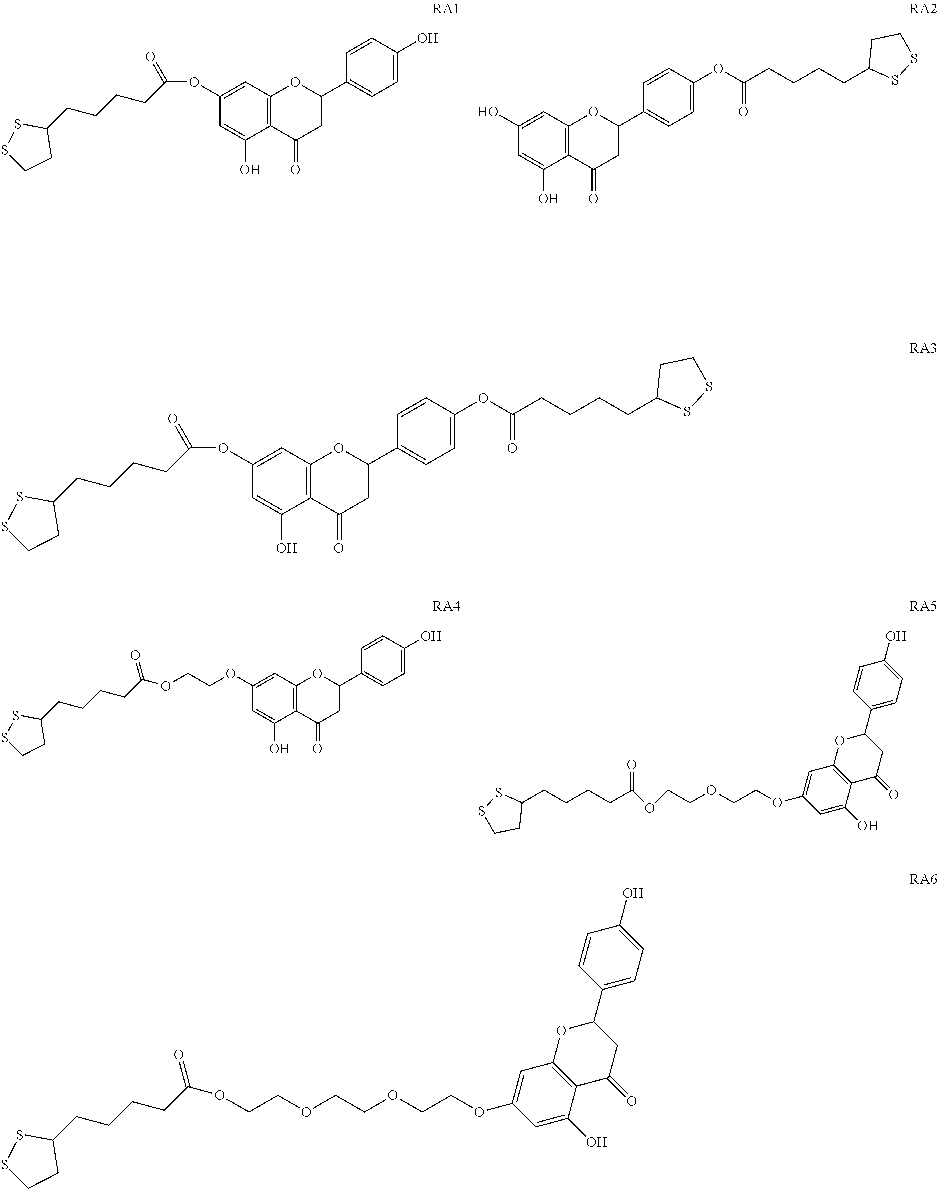 [SMILES: O=C(CCCCC1CCSS1)Oc1cc(O)c2c(c1)OC(c1ccc(O)cc1)CC2=O ; O=C(CCCCC1CCSS1)Oc1ccc(C2CC(=O)c3c(O)cc(O)cc3O2)cc1 ; O=C(CCCCC1CCSS1)Oc1ccc(C2CC(=O)c3c(O)cc(OC(=O)CCCCC4CCSS4)cc3O2)cc1 ; O=C(CCCCC1CCSS1)OCCOc1cc(O)c2c(c1)OC(c1ccc(O)cc1)CC2=O ; O=C(CCCCC1CCSS1)OCCOCCOc1cc(O)c2c(c1)OC(c1ccc(O)cc1)CC2=O ; O=C(CCCCC1CCSS1)OCCOCCOCCOc1cc(O)c2c(c1)OC(c1ccc(O)cc1)CC2=O]

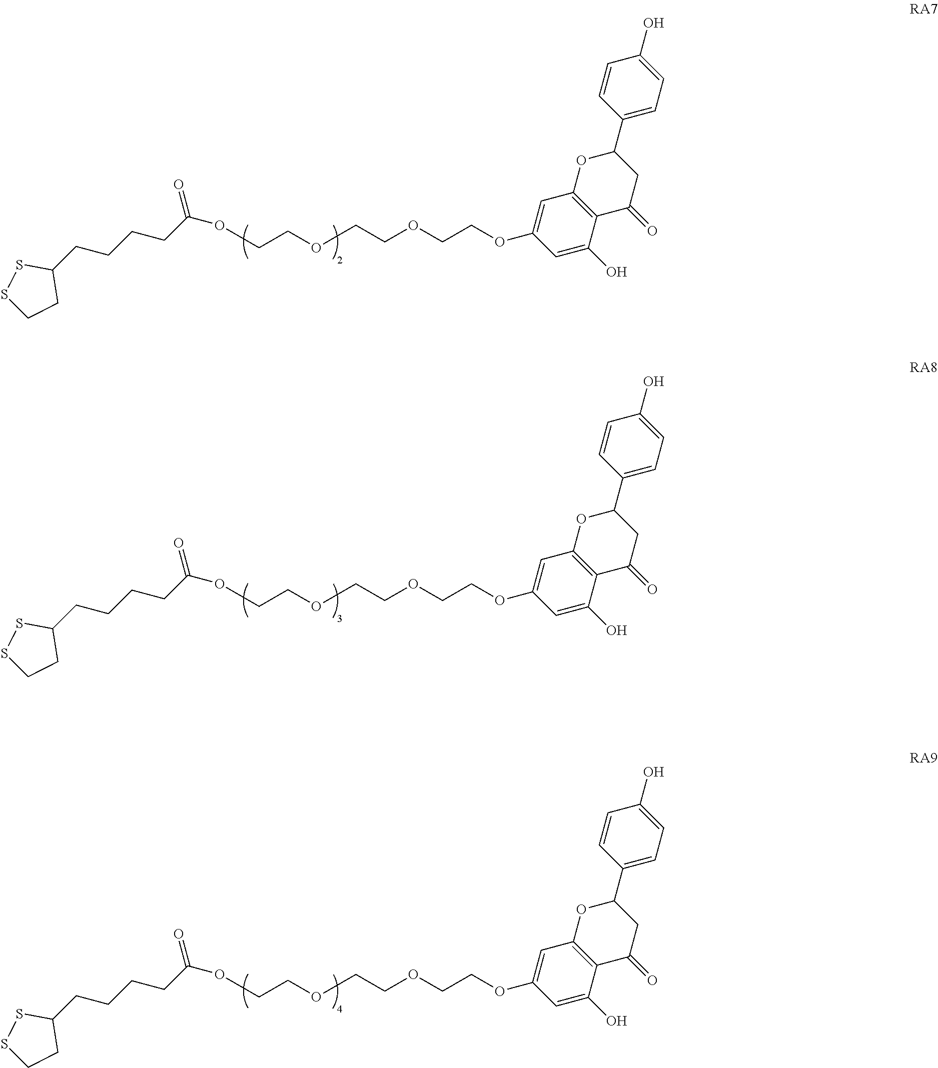

In addition, the present invention provides a pharmaceutical composition for treatment or prevention of stroke, cerebral infarction, cerebral hemorrhage, cognitive impairment, Parkinson's disease, Alzheimer's disease, vascular dementia, memory loss, cerebral nerve injury, etc., comprising the compound according to the present invention or a pharmaceutical salt thereof as an active ingredient.

In the pharmaceutical composition of the present invention, the compound according to the present invention may be administered in an appropriate formulation together with a carrier and a diluent known in the art, and orally or parenterally administered according to a desired method, and for example, may have formulations such as intravenous injection, intramuscular injection, intraperitoneal injection, subcutaneous injection, and suppositories.

The formulations may be prepared by a general method using suitable excipients, fillers, binders, wetting agents, disintegrants, lubricants, surfactants, dispersants, buffers, preservatives, solubilizers, disinfectants, sweeteners, spices, analgesics, stabilizers, isotonic solutions, which have been commonly used in a pharmaceutical composition.

Each of the formulations described above may contain a pharmaceutically acceptable carrier or additive. Specific examples of the carrier or additive include water, pharmaceutically acceptable organic solvents, collagen, polyvinyl alcohol, polyvinylpyrrolidine, carboxyvinyl polymer, sodium alginate, water-soluble dextran, carboxymethyl sodium starch, pectin, xanthan rubber, arabic rubber, casein, gelatin, agar, glycerol, propylene glycol, polyethyl glycol, petrolatum, paraffin, stearyl alcohol, stearic acid, human serum albumin, mannitol, sorbitol, lactic acid, etc. One or more additives may be selected or appropriately combined depending on a form of the formulation. Furthermore, as a method of administering a cell therapeutic agent, local administration to target cells may be performed in addition to conventional systemic administration such as intravenous or intraarterial administration, and an administration method combined with catheter technology and surgical surgery may be used.

The composition of the present invention may contain the compound according to the present invention in a pharmaceutically effective dose together with a pharmaceutically acceptable carrier.

In the present invention, the term “pharmaceutically effective dose” refers to an amount of an active ingredient that exhibits effects of alleviating, inhibiting, ameliorating and/or treating immune rejection disease to be treated. The dose of the compound according to the present invention varies in the range thereof according to the patient's weight, age, sex, health condition, diet, administration time, administration method, the severity of disease, etc. For example, a therapeutically effective dose may be initially determined using an in vitro assay via cell culture. Without excessive experiments in the art, an effective dose for treatment may be determined, and a useful dose in the human may be more accurately determined using this information. For example, the compound according to the present invention may be administered as an active ingredient in an amount of 0.1 to 100 mg/kg/day.

The individual in need of the treatment may be mammals, for example, humans.

In addition, the present invention provides a functional health food for improvement or prevention of stroke, cerebral infarction, cerebral hemorrhage, cognitive impairment, Parkinson's disease, Alzheimer's disease, vascular dementia, memory loss, cerebral nerve injury, etc., comprising the compound according to the present invention or a pharmaceutical salt thereof as an active ingredient.

The term “food” means natural products or processed products containing one or more nutrients, and preferably, means a condition that may be eaten directly through a certain amount of processing process, and as a general meaning, may mean including all of foods, food additives, functional foods and beverages.

Foods to which the composition for food may be added include, for example, various foods, beverages, gum, tea, vitamin complexes, and functional foods. In addition, the foods include special nutritional foods (e.g., milk formulas, infant/baby food, etc.), processed meat products, fish meat products, tofu, jelly, noodles (e.g., ramen, noodles, etc.), bread, health supplements, seasoning foods (e.g., soy sauce, soybean paste, red pepper paste, mixed sauce, etc.), sauces, confectionery (e.g. snacks), candies, chocolates, gums, ice creams, dairy products (e.g. fermented milk, cheese, etc.), other processed foods, kimchi, pickles (various kimchis, pickles, etc.), beverages (e.g., fruit beverages, vegetable beverages, soy milk, fermented beverages, etc.), and natural seasonings (e.g., ramen soup, etc.), but are not limited thereto. The foods, beverages or food additives may be prepared by general preparation methods.

In addition, the term “functional food” or “health functional food” refers to a group of foods that have added value to the food to act and express the function of the food for a specific purpose by using physical, biochemical, and bioengineering techniques, or a food that is designed and processed to sufficiently express body modulating functions for biological defense rhythm control, disease prevention and recovery, etc. of the food composition. Specifically, the “functional food” or “health functional food” may be a health functional food. The functional food may include food-acceptable food supplement additives, and may further include suitable carriers, excipients, and diluents which are commonly used in the preparation of functional foods.

The type of health supplement food is not limited thereto, but may be in the form of powders, granules, tablets, capsules or beverages.

Further, the present invention provides a skin external preparation composition comprising the compound according to the present invention or a pharmaceutical salt thereof as an active ingredient.

The skin external preparation composition may have anti-aging effects, such as UV protection, antioxidation, whitening, wrinkle relief, anti-inflammatory, acne, production promotion and decomposition prevention of collagen and elastin production, or removal of active oxygen.

The skin external preparation composition may be in the form of oil-in-water (O/W) or water-in-oil (W/O) including gels, packs, cream, essence, lotion, toner, and surfactants, but is not limited thereto.

Further, the present invention provides a method for preparing the compound according to the present invention comprising reacting naringenin and lipoic acid.

In one embodiment of the present invention, the compound may be prepared by directly chemically covalently bonding lipoic acid and naringenin by selecting an appropriate coupling reagent. The coupling reagent includes, for example, dimethylaminopyridine (DMAP), dicyclohexylcarbodiimide (DCC), and the like, but is not limited thereto.

In one embodiment of the present invention, the lipoic acid and the naringenin may be chemically covalently bonded through a linker. Specifically, several novel compounds having different binding positions may be prepared by selectively reacting one of hydroxyl groups of naringenin reacting with lipoic acid, and in particular, various compound structures may be prepared through a multi-step reaction using a linker having a variety of carbon atoms in ether. The linker may include, for example, —OCH₂CH₂—, —OCH₂CH₂OCH₂CH₂—, —OCH₂CH₂OCH₂CH₂OCH₂CH₂—, —OCH₂CH₂OCH₂CH₂OCH₂CH₂OCH₂CH₂—, —OCH₂CH₂OCH₂CH₂OCH₂CH₂OCH₂CH₂OCH₂CH₂— and —OCH₂CH₂OCH₂CH₂OCH₂CH₂OCH₂CH₂OCH₂CH₂OCH₂CH₂—.

Hereinafter, the present invention will be described in detail by Examples. However, these Examples are only illustrative of the present invention, and the scope of the present invention is not limited to these Examples.

### EXAMPLES

### Example 1

### Preparation of [5-hydroxy-2-(4-hydroxyphenyl)-4-oxochroman-7-yl 5-(1,2-dithiolan-3-yl)pentanoate] (RA1)

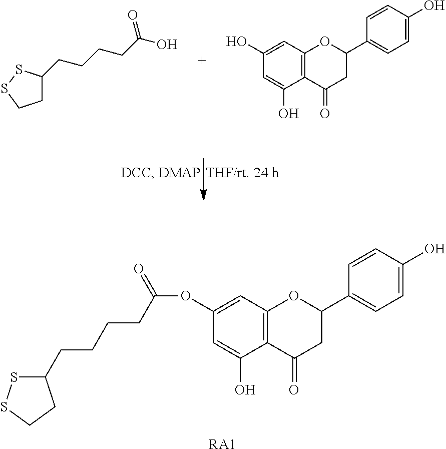

Lipoic acid (2.0 mmol) and naringenin (2.0 mmol) were dissolved in THF (15 mL), and then dimethylaminopyridine (DMAP: 2.0 mmol) and dicyclohexylcarbodiimide (DCC: 2.0 mmol) were added to the solution. After stirring at room temperature for 24 hours, the reaction was terminated. The produced and precipitated urea was filtered and a filtrate was concentrated. The residue was dissolved in MC again, the solution was treated with 10% citric acid and brine, and then water was removed with anhydrous MgSO₄. After the solvent was removed with a concentrator, the product was subjected to column chromatography using a developing solution (ethyl acetate:nucleic acid=1:2) to obtain a pure novel compound RA1 in yield of 52%.

¹HNMR (400 MHz, CDCl₃) d7.46 (d, 2H, 8.4 Hz), 7.15 (d, 2H, 8.4 Hz), 6.60 (s, 1H), 6.01-5.98 (m, 2H), 5.42 (dd, 1H, 12.9 Hz, 3.0 Hz), 3.61 (m, 1H), 3.15 (m, 2H), 3.03 (dd, 1H, 14.0 Hz, 12.0 Hz), 2.81 (dd, 1H, 14.0 Hz, 4.0 Hz), 2.60 (t, 2H, 7.50 Hz), 2.48-2.42 (m, 2H), 1.65-1.52 (m, 4H), 1.50-1.46 (m, 2H); MS (ESI): m/z 461.17 (M⁺+1); Anal. Calcd. For C₂₃H₂₄O₆S₂: C, 59.98; H, 5.25. Found: C, 59.81; H, 5.10.

### Example 2

### Preparation of [4-(5,7-dihydroxy-4-oxochroman-2-yl)phenyl 5-(1,2-dithiolan-3-yl)pentanoate] (RA2)

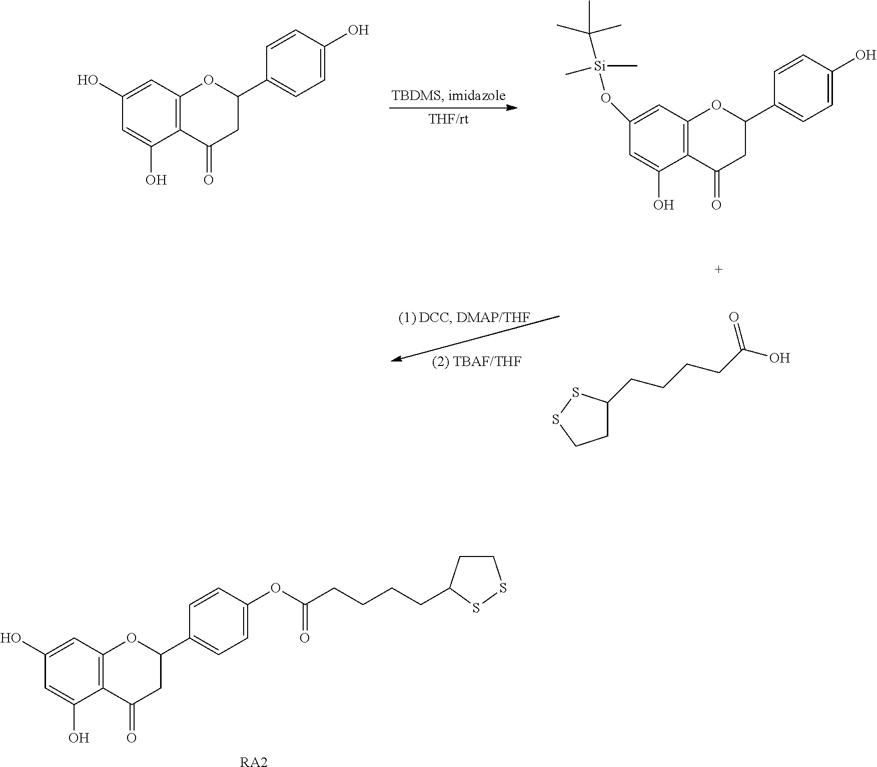

Naringenin (2.0 mmol), tert-butyldimethylsilyl chloride (TBDMS-Cl: 2.3 mmol), and imidazole (2.2 mmol) were added and dissolved in THF (15 mL) and then reacted at room temperature. After termination of the reaction, water and MC were added, and an organic layer was separated and concentrated. Herein, THF (15 mL), lipoic acid (2.0 mmol), dimethylamino-pyridine (DMAP: 2.0 mmol), and dicyclohexylcarbodiimide (DCC: 2.0 mmol) were added again to the solution and stirred at room temperature for 24 hours to terminate the reaction. The produced and precipitated urea was filtered and a filtrate was concentrated. The residue was dissolved in water and MC again, the solution was treated with 10% citric acid and brine, and then the water was removed with anhydrous MgSO₄. After the solvent was removed with a concentrator, THF (15 mL) and tetrabutylammonium fluoride (TBAF: 2.0 mmol) were again added thereto, reacted, and monitored by TLC. Water and MC were added, the solution was treated with 10% citric acid and brine, and then the water was removed with anhydrous MgSO₄. After the solvent was removed with a concentrator, the product was subjected to column chromatography using a developing solution (ethyl acetate:nucleic acid=1:2) to obtain a pure novel compound RA2 in yield of 40%.

¹HNMR (400 MHz, CDCl₃) d7.40 (d, 2H, 8.4 Hz), 7.10 (d, 2H, 8.4 Hz), 6.62 (s, 1H), 6.01-5.96 (m, 2H), 5.40 (dd, 1H, 12.9 Hz, 3.0 Hz), 3.59 (m, 1H), 3.10 (m, 2H), 3.00 (dd, 1H, 14.0 Hz, 12.0 Hz), 2.80 (dd, 1H, 14.0 Hz, 4.0 Hz), 2.63 (t, 2H, 7.50 Hz), 2.50-2.41 (m, 2H), 1.68-1.45 (m, 4H), 1.40-1.35 (m, 2H); MS (ESI): m/z 461.33 (M⁺+1); Anal. Calcd. For C₂₃H₂₄O₆S₂: C, 59.98; H, 5.25. Found: C, 59.86; H, 5.12.

### Example 3

### Preparation of [4-(7-((5-(1,2-dithiolan-3-yl)pentanoyl)oxy)-5-hydroxy-4-oxochroman-2-yl)phenyl 5-(1,2-dithiolan-3-yl)pentanoate] (RA3)

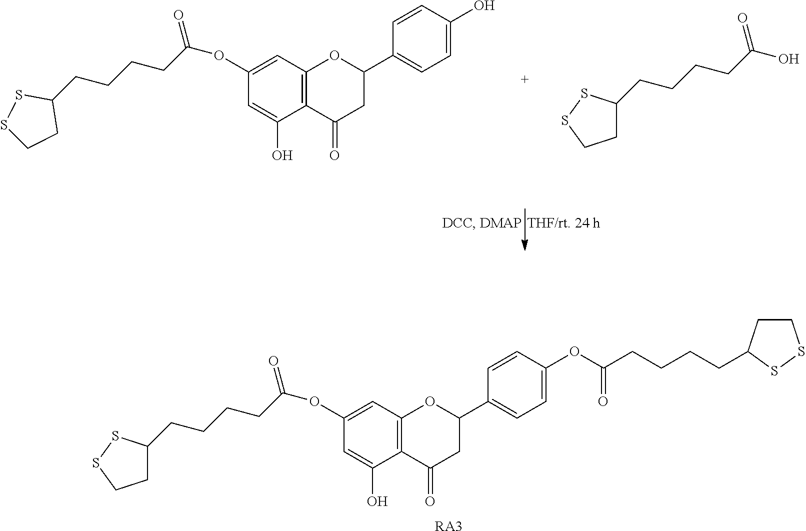

Lipoic acid (2.0 mmol) and RA1 (2.0 mmol) were dissolved in THF (15 mL), and then dimethylaminopyridine (DMAP: 2.0 mmol) and dicyclohexylcarbodiimide (DCC: 2.0 mmol) were added to the solution and stirred at room temperature for 24 hours to terminate the reaction. The produced and precipitated urea was filtered and a filtrate was concentrated. The residue was dissolved in MC again, the solution was treated with brine, and then water was removed with anhydrous MgSO₄. After the solvent was removed with a concentrator, the product was subjected to column chromatography using a developing solution (ethyl acetate:nucleic acid=1:2) to obtain a pure novel compound RA3 in yield of 44%.

¹HNMR (400 MHz, CDCl₃) d7.40 (dd, 4H, 8.4 Hz), 7.22 (dd, 4H, 8.4 Hz), 6.62 (s, 2H), 6.15-5.90 (m, 4H), 5.40 (dd, 2H), 3.59-3.57 (m, 2H), 3.10 (m, 4H), 3.00 (dd, 2H), 2.83 (dd, 2H), 2.63 (m, 4H), 2.55-2.45 (m, 4H), 1.65-1.40 (m, 8H), 1.40-1.30 (m, 4H); MS (ESI): m/z 649.47 (M⁺+1); Anal. Calcd. For C₃₁H₃₆O₇S₄: C, 57.38; H, 5.59. Found: C, 57.59; H, 5.66.

### Example 4

### Preparation of [2-((5-hydroxy-2-(4-hydroxyphenyl)-4-oxochroman-7-yl)oxy)ethyl 5-(1,2-dithiolan-3-yl)pentanoate] (RA4)

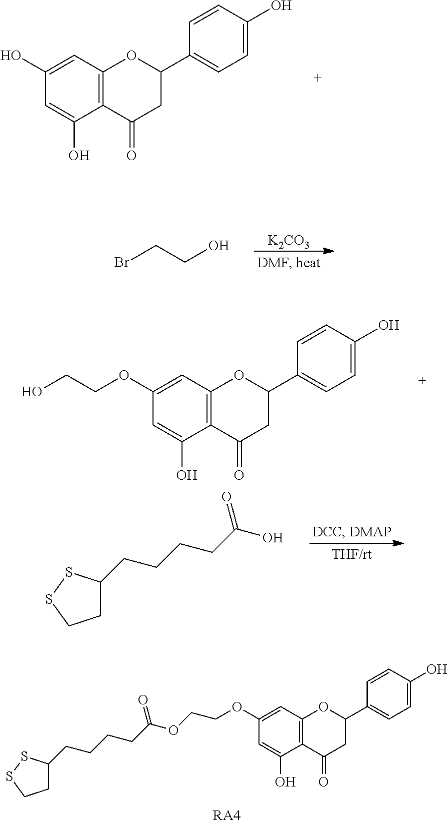

Naringenin (2.0 mmol), bromoethanol (2.3 mmol), and potassium carbonate (2.5 mmol) were added and dissolved in THF (15 mL) and then refluxed and reacted. After termination of the reaction, water and MC were added, and an organic layer was separated and concentrated. Herein, THF (15 mL), lipoic acid (2.0 mmol), dimethylamino-pyridine (DMAP: 2.0 mmol), and dicyclohexylcarbodiimide (DCC: 2.0 mmol) were added again to the solution and stirred at room temperature for 24 hours to terminate the reaction. The produced and precipitated urea was filtered and a filtrate was concentrated. Water and MC were added, the solution was treated with 10% citric acid and brine, and then the water was removed with anhydrous MgSO₄. After the solvent was removed with a concentrator, the product was subjected to column chromatography using a developing solution (ethyl acetate:nucleic acid=1:2) to obtain a pure novel compound RA4 in yield of 48%.

¹HNMR (400 MHz, CDCl₃) d7.40 (d, 2H, 8.4 Hz), 7.14 (d, 2H, 8.4 Hz), 6.62 (s, 1H), 6.01-5.97 (m, 2H), 5.40 (dd, 1H, 12.9 Hz, 3.0 Hz), 4.50-4.40 (m, 4H), 3.63 (m, 1H), 3.16 (m, 2H), 3.00 (dd, 1H, 14.0 Hz, 12.0 Hz), 2.82 (dd, 1H, 14.0 Hz, 4.0 Hz), 2.62 (t, 2H, 7.50 Hz), 2.47-2.40 (m, 2H), 1.68-1.50 (m, 4H), 1.50-1.42 (m, 2H); MS (ESI): m/z 505.22 (M⁺+1); Anal. Calcd. For C₂₅H₂₈O₇S₂: C, 59.50; H, 5.59. Found: C, 59.80; H, 5.49.

### Example 5

### Preparation of [2-(2-((5-hydroxy-2-(4-hydroxyphenyl)-4-oxochroman-7-yl)oxy)ethoxy) ethyl 5-(1,2-dithiolan-3-yl)pentanoate) (RA5)

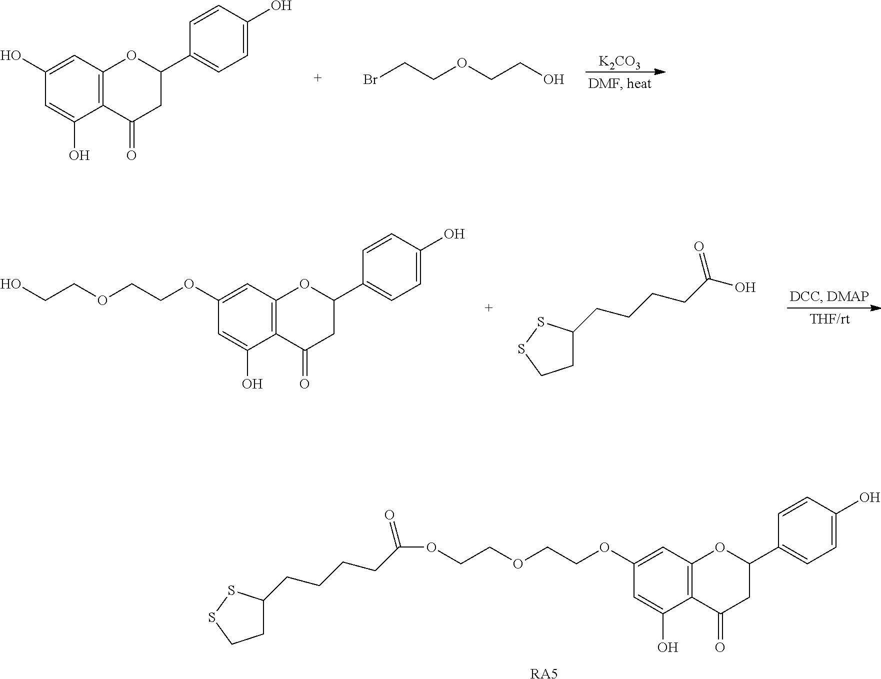

Naringenin (2.0 mmol), 2-(2-bromoethoxy)ethanol (2.3 mmol), and potassium carbonate (2.5 mmol) were added and dissolved in THF (15 mL) and then refluxed and reacted. After termination of the reaction, water and MC were added, and an organic layer was separated and concentrated. Herein, THF (15 mL), lipoic acid (2.0 mmol), dimethylamino-pyridine (DMAP: 2.0 mmol), and dicyclohexylcarbodiimide (DCC: 2.0 mmol) were added again to the solution and stirred at room temperature for 24 hours to terminate the reaction. The produced and precipitated urea was filtered and a filtrate was concentrated. Water and MC were added, the solution was treated with 10% citric acid and brine, and then the water was removed with anhydrous MgSO₄. After the solvent was removed with a concentrator, the product was subjected to column chromatography using a developing solution (ethyl acetate:nucleic acid=1:2) to obtain a pure novel compound RA5 in yield of 38%.

¹HNMR (400 MHz, CDCl₃) d7.44 (d, 2H, 8.4 Hz), 7.13 (d, 2H, 8.4 Hz), 6.60 (s, 1H), 6.08-6.00 (m, 2H), 5.42 (dd, 1H, 12.9 Hz, 3.0 Hz), 4.50-4.42 (m, 4H), 4.30 (t, 2H), 4.21 (t, 2H), 3.80 (t, 2H), 3.63-3.60 (m, 3H), 3.18 (m, 2H), 3.05 (dd, 1H, 14.0 Hz, 12.0 Hz), 2.80 (dd, 1H, 14.0 Hz, 4.0 Hz), 2.60 (t, 2H, 7.50 Hz), 2.49-2.42 (m, 2H), 1.68-1.55 (m, 4H), 1.52-1.44 (m, 2H); MS (ESI): m/z 549.55 (M⁺+1); Anal. Calcd. For C₂₇H₃₂O₈S₂: C, 59.10; H, 5.88. Found: C, 59.33; H, 5.72.

### Example 6

### Preparation of [2-(2-(2-((5-hydroxy-2-(4-hydroxyphenyl)-4-oxochroman-7-yl)oxy)ethoxy)ethoxy)ethyl 5-(1,2-dithiolan-3-yl)pentanoate] (RA6)

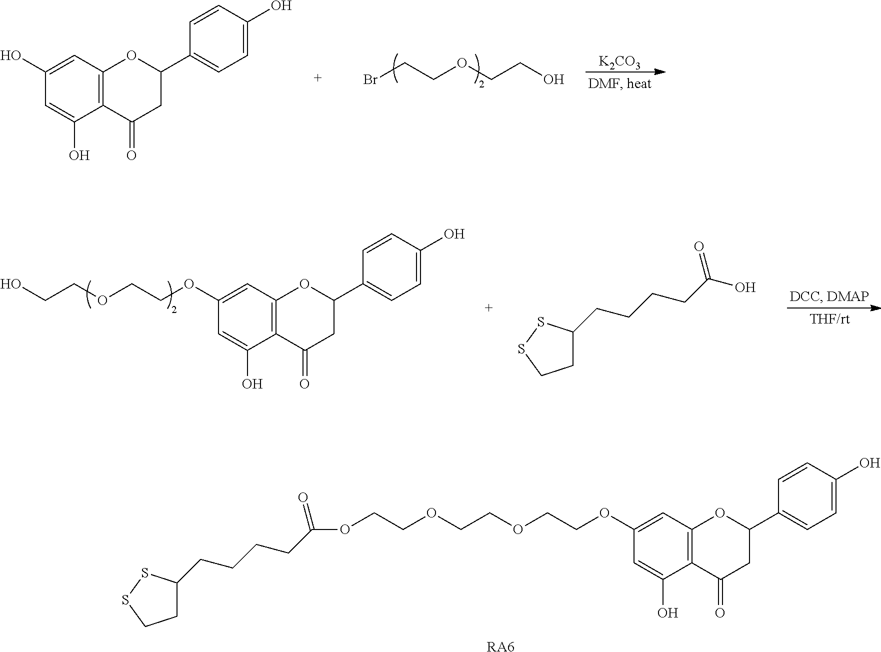

Naringenin (2.0 mmol), 2-(2-(2-bromoethoxy)ethoxy)ethanol (2.3 mmol), and potassium carbonate (2.5 mmol) were added and dissolved in THF (15 mL) and then refluxed and reacted. After termination of the reaction, water and MC were added, and an organic layer was separated and concentrated. Herein, THF (15 mL), lipoic acid (2.0 mmol), dimethylamino-pyridine (DMAP: 2.0 mmol), and dicyclohexylcarbodiimide (DCC: 2.0 mmol) were added again to the solution and stirred at room temperature for 24 hours to terminate the reaction. The produced and precipitated urea was filtered and a filtrate was concentrated. Water and MC were added, the solution was treated with 10% citric acid and brine, and then the water was removed with anhydrous MgSO₄. After the solvent was removed with a concentrator, the product was subjected to column chromatography using a developing solution (ethyl acetate:nucleic acid=1:2) to obtain a pure novel compound RA6 in yield of 35%.

¹HNMR (400 MHz, CDCl₃) d7.40 (d, 2H, 8.4 Hz), 7.15 (d, 2H, 8.4 Hz), 6.58 (s, 1H), 6.11-6.06 (m, 2H), 5.40 (dd, 1H, 12.9 Hz, 3.0 Hz), 4.52-4.44 (m, 4H), 4.30 (t, 2H), 4.20 (t, 2H), 3.83 (t, 2H), 3.63-3.59 (m, 3H), 3.54 (s, 4H), 3.16 (m, 2H), 3.08 (dd, 1H, 14.0 Hz, 12.0 Hz), 2.83 (dd, 1H, 14.0 Hz, 4.0 Hz), 2.62 (t, 2H, 7.50 Hz), 2.49-2.45 (m, 2H), 1.66-1.58 (m, 4H), 1.54-1.45 (m, 2H); MS (ESI): m/z 593.82 (M⁺+1); Anal. Calcd. For C₂₉H₃₆O₉S₂: C, 58.76; H, 6.12. Found: C, 58.59; H, 6.01.

### Example 7

### Preparation of [2-(2-(2-(2-((5-hydroxy-2-(4-hydroxyphenyl)-4-oxochroman-7-yl)oxy)ethoxy)ethoxy)ethoxy)ethyl 5-(1,2-dithiolan-3-yl)pentanoate](RA7)

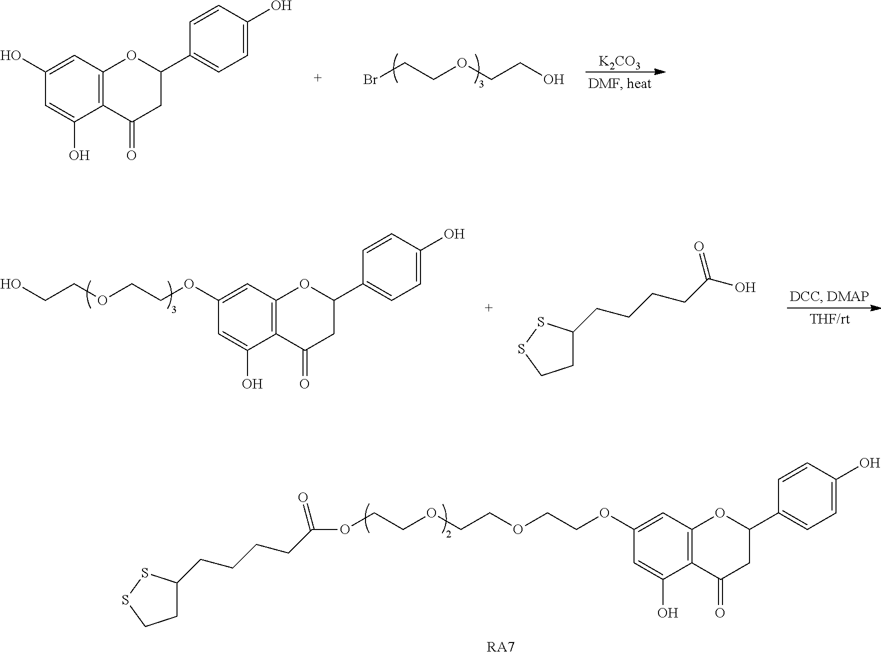

Naringenin (2.0 mmol), 2-(2-(2-(2-bromoethoxy)ethoxy)ethoxy)ethanol (2.3 mmol), and potassium carbonate (2.5 mmol) were added and dissolved in THF (20 mL) and then refluxed and reacted. After termination of the reaction, water and MC were added, and an organic layer was separated and concentrated. Herein, THF (15 mL), lipoic acid (2.0 mmol), dimethylamino-pyridine (DMAP: 2.0 mmol), and dicyclohexylcarbodiimide (DCC: 2.0 mmol) were added again to the solution and stirred at room temperature for 24 hours to terminate the reaction. The produced and precipitated urea was filtered and a filtrate was concentrated. Water and MC were added, the solution was treated with 10% citric acid and brine, and then the water was removed with anhydrous MgSO₄. After the solvent was removed with a concentrator, the product was subjected to column chromatography using a developing solution (ethyl acetate:nucleic acid=1:1) to obtain a pure novel compound RA7 in yield of 30%.

¹HNMR (400 MHz, CDCl₃) d7.38 (d, 2H, 8.4 Hz), 7.17 (d, 2H, 8.4 Hz), 6.59 (s, 1H), 6.10-6.05 (m, 2H), 5.42 (dd, 1H, 12.9 Hz, 3.0 Hz), 4.52-4.42 (m, 4H), 4.32 (t, 2H), 4.21 (t, 2H), 3.83 (t, 2H), 3.65-3.62 (m, 3H), 3.53 (m, 8H), 3.18 (m, 2H), 3.09 (dd, 1H, 14.0 Hz, 12.0 Hz), 2.85 (dd, 1H, 14.0 Hz, 4.0 Hz), 2.60 (t, 2H, 7.50 Hz), 2.48-2.44 (m, 2H), 1.68-1.57 (m, 4H), 1.52-1.49 (m, 2H); MS

(ESI): m/z 637.49 (M⁺+1); Anal. Calcd. For C₃₁H₄₀O₁₀S₂: C, 58.47; H, 6.33. Found: C, 58.60; H, 6.24.

### Example 8

### Preparation of [14-((5-hydroxy-2-(4-hydroxyphenyl)-4-oxochroman-7-yl)oxy)-3,6,9,12-tetraoxatetradecyl 5-(1,2-dithiolan-3-yl)pentanoate] (RA8)

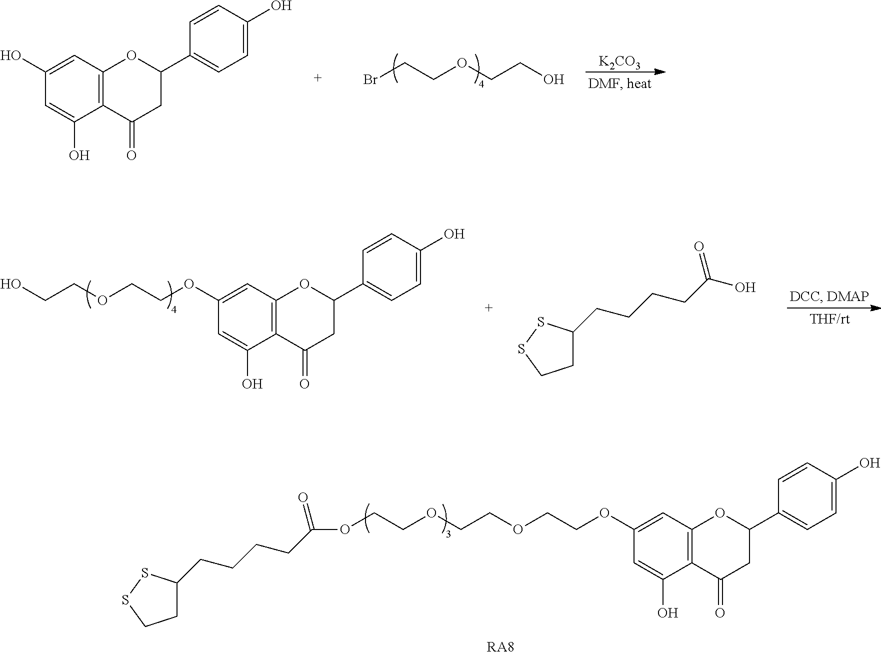

Naringenin (2.0 mmol), 14-bromo-3,6,9,12-tetraoxatetradecan-1-ol (2.3 mmol), and potassium carbonate (2.5 mmol) were added and dissolved in THF (20 mL) and then refluxed and reacted. After termination of the reaction, water and MC were added, and an organic layer was separated and concentrated. Herein, THF (15 mL), lipoic acid (2.0 mmol), dimethylamino-pyridine (DMAP: 2.0 mmol), and dicyclohexylcarbodiimide (DCC: 2.0 mmol) were added again to the solution and stirred at room temperature for 24 hours to terminate the reaction. The produced and precipitated urea was filtered and a filtrate was concentrated. Water and MC were added, the solution was treated with 10% citric acid and brine, and then the water was removed with anhydrous MgSO₄. After the solvent was removed with a concentrator, the product was subjected to column chromatography using a developing solution (ethyl acetate:nucleic acid=1:1) to obtain a pure novel compound RA8 in yield of 35%.

¹HNMR (400 MHz, CDCl₃) d7.34 (d, 2H, 8.4 Hz), 7.15 (d, 2H, 8.4 Hz), 6.57 (s, 1H), 6.12-6.06 (m, 2H), 5.40 (dd, 1H, 12.9 Hz, 3.0 Hz), 4.50-4.42 (m, 4H), 4.32 (t, 2H), 4.20 (t, 2H), 3.85 (t, 2H), 3.68-3.65 (m, 3H), 3.53 (m, 12H), 3.18 (m, 2H), 3.05 (dd, 1H, 14.0 Hz, 12.0 Hz), 2.88 (dd, 1H, 14.0 Hz, 4.0 Hz), 2.62 (t, 2H, 7.50 Hz), 2.46-2.42 (m, 2H), 1.68-1.55 (m, 4H), 1.54-1.49 (m, 2H); MS

(ESI): m/z 681.34 (M⁺+1); Anal. Calcd. For C₃₃H₄₄O₁₁S₂: C, 58.22; H, 6.51. Found: C, 58.10; H, 6.45.

### Example 9

### Preparation of [17-((5-hydroxy-2-(4-hydroxyphenyl)-4-oxochroman-7-yl)oxy)-3,6,9,12,15-pentaoxaheptadecyl 5-(1,2-dithiolan-3-yl)pentanoate] (RA9)

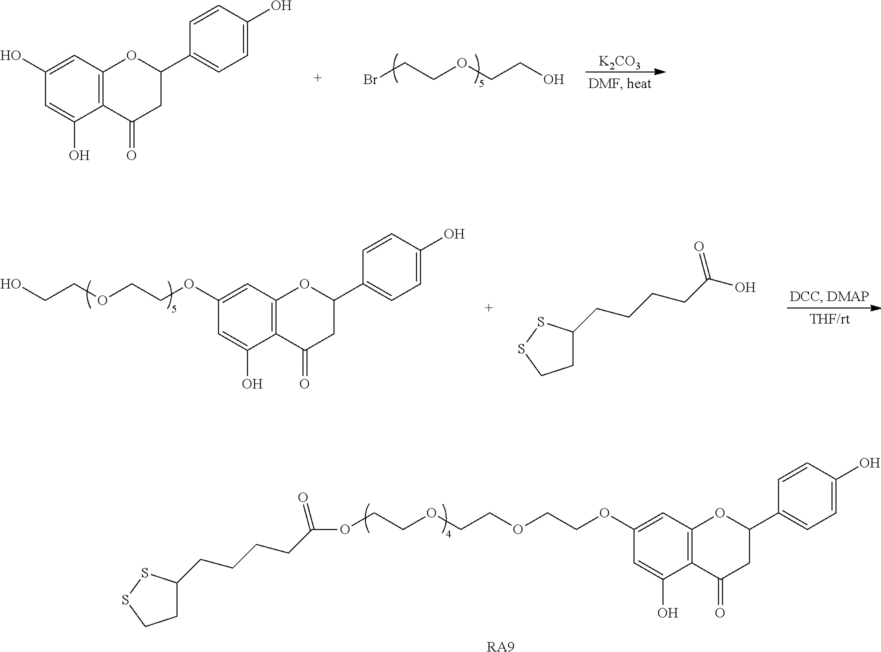

Naringenin (2.0 mmol), 17-bromo-3,6,9,12,15-pentaoxaheptadecan-1-ol (2.3 mmol), and potassium carbonate (2.5 mmol) were added and dissolved in THF (20 mL) and then refluxed and reacted. After termination of the reaction, water and MC were added, and an organic layer was separated and concentrated. Herein, THF (15 mL), lipoic acid (2.0 mmol), dimethylamino-pyridine (DMAP: 2.0 mmol), and dicyclohexylcarbodiimide (DCC: 2.0 mmol) were added again to the solution and stirred at room temperature for 24 hours to terminate the reaction. The produced and precipitated urea was filtered and a filtrate was concentrated. Water and MC were added, the solution was treated with 10% citric acid and brine, and then the water was removed with anhydrous MgSO₄. After the solvent was removed with a concentrator, the product was subjected to column chromatography using a developing solution (ethyl acetate:nucleic acid=1:1) to obtain a pure novel compound RA9 in yield of 31%.

¹HNMR (400 MHz, CDCl₃) d7.46 (d, 2H, 8.4 Hz), 7.13 (d, 2H, 8.4 Hz), 6.60 (s, 1H), 6.02-5.95 (m, 2H), 5.40 (dd, 1H, 12.9 Hz, 3.0 Hz), 4.50-4.42 (m, 4H), 4.30 (t, 2H), 4.22 (t, 2H), 3.88 (t, 2H), 3.68-3.62 (m, 3H), 3.50 (m, 20H), 3.20 (m, 2H), 3.06 (dd, 1H, 14.0 Hz, 12.0 Hz), 2.80 (dd, 1H, 14.0 Hz, 4.0 Hz), 2.64 (t, 2H, 7.50 Hz), 2.48-2.44 (m, 2H), 1.68-1.50 (m, 6H); MS (ESI): m/z 725.80 (M⁺+1); Anal. Calcd. For C₃₅H₄₈O₁₂S₂: C, 57.99; H, 6.67. Found: C, 58.19; H, 6.50.

### Experimental Examples

In order to confirm an effect of preventing and treating, particularly, cerebral infarction among the biological activities of the compounds prepared in the present invention, brain cell culture, oxygen-glucose deprivation/reoxygenation (OGD/R), cytotoxicity, total antioxidant capacity, etc. were examined as cell tests.

In animal experiments, transient middle cerebral artery occlusion (t-MCAO) was used to temporally test the protective efficacy of the cerebral infarction site.

In addition, an antioxidant effect, a tyrosinase inhibitory effect, etc. were tested to confirm the efficacy of the compounds as a skin external preparation.

### Experimental Example 1

In Vitro Cell Cultures

A renal cortex culture medium containing neurons and glia was obtained from fetal rats during the gestation period of 17 to 18 days. That is, an embryonic tissue was extracted from a pregnant Sprague-Dawley and transferred to a Hanks Balanced Salt solution (Gibco) cooled with ice. The cortical brain tissue was sterile and thinly dissected and stored in cold HBSS before digestion with 0.0125% trypsin.

The isolated cells were placed in a 96 well plate coated with poly-L-lysine (1 mg/ml, Sigma-Aldrich) in a Dulbecco's modified eagle medium (Gibco) containing 10% iron-supplemented fetal calf serum (Hyclone) and 1% antibiotic/antimycotic (Gibco) at 37° C. at a seeding density of 50,000 cells/well. Then, after attaching the culture medium overnight in a 37° C. incubator containing 5% CO₂ and atmospheric oxygen, at the next day, the medium was replaced with a warm Neurobasal A medium (Gibco) containing 0.5 mmol/L of L-glutamine (Gibco) and supplemented with 1% B27, N2 and antibiotics/antimycotic (Gibco). In the culture for 14 to 16 days, half of the medium was changed every 4 days.

### Experimental Example 2

Brain Cell Protection Effect of Naringenin, Lipoic Acid, or RA1 (VANL-100) in Apoptosis Induced by Oxygen-Glucose Deprivation (OGD)

After 14 days of cell culture, the culture medium was replaced with a vehicle (0.1% DMSO or EtOH) or an oxygen-free-glucose Neurobasal A containing an experimental substance (RA1, naringenin or lipoic acid) at 6 concentrations. After oxygen-glucose deprivation (OGD), the same volume of Neurobasal A with the same novel substance concentration was added and transferred to a normoxic condition (5% CO₂ and atmospheric oxygen) state for 24 hours. Cellular activity was evaluated by measuring LDH release of damaged cells.

The effects of lipoic acid, naringenin, and the novel substance RA1 on apoptosis (% LDH release) following ischemia/reoxygenation-reperfusion (I/R) were performed in the in vitro cell culture.

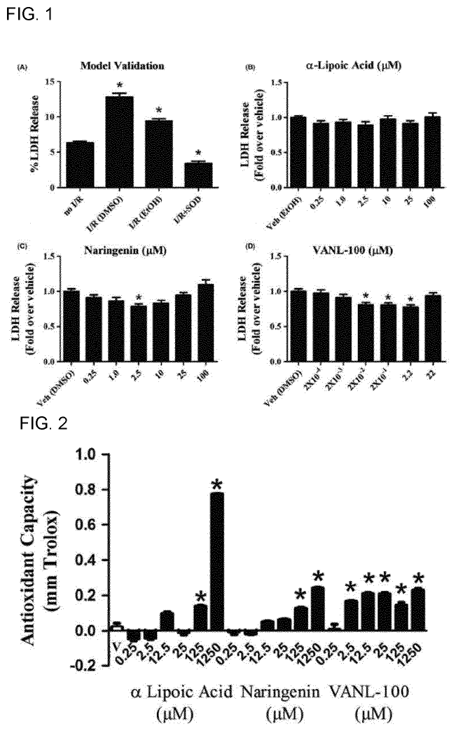

As illustrated in FIG. 1, lipoic acid (B) hardly showed a protective effect on brain cells, and naringenin (C) had a slight effect at one concentration. On the other hand, RA1 (VANL-100) showed a very strong protective effect on brain cells of about 100 times at a very low concentration, as illustrated in FIG. 1D.

### Experimental Example 3

Total Antioxidant Capacity Assay of Lipoic Acid, Naringenin, and RA1 (VANL-100)

Several groups of plates were subjected to the OGD (oxygen-glucose deprivation) and I/R (ischemia-reperfusion), and the total antioxidant capacity was measured using a general commercial instrument (Antioxidant Assay Kit, Sigma-Aldrich). The average value of the three experiments was obtained and the antioxidant capacity was expressed in mm Trolox.

As Illustrated in FIG. 2, lipoic acid had no antioxidant effect at concentrations of 0.25 and 25 ummol/L, but showed a great antioxidant capacity at high concentrations of 125 and 1250 ummol/L. Naringenin also showed a similar degree of protective effect on brain cells at high concentrations of 125 and 1250 ummol/L. However, RA1 (VANL-100) showed a strong protective effect on brain cells at a broad concentration of 2.5 to 1250 ummol/L.

### Experimental Example 4

Effect of Pre-Administration of RA1 (VANL-100) and Naringenin on Cerebral Infarction Site Using Transient Middle Cerebral Artery Occlusion (t-MCAO) in Animal Experiments

As a research model for cerebral infarction, transient middle cerebral artery occlusion (t-MCAO) in animals (rats) was used to examine the infarct size of the cerebral infarction site and the degree of its protective action.

First, in an animal model in which the middle cerebral artery of Sprague-Dawley rats (300 to 350 g; Charles River, Montreal, PQ, CAN) was closed for 30 minutes to create a cerebral infarction and then reperfused for 5 hours and 30 minutes (t-MCAO), naringenin and RA1 (VANL-100) were administered immediately before artery occlusion and compared with a case of administration of a vehicle.

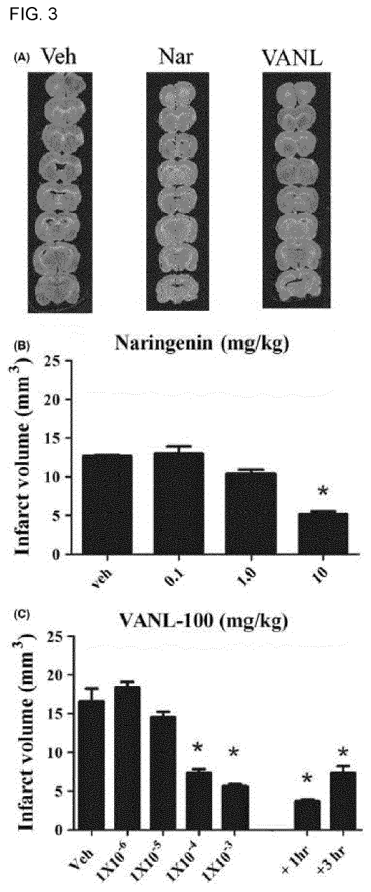

As a result, as shown in a photograph (A) of FIG. 3, when naringenin or RA1 was administered, the size of the cerebral infarction site was significantly reduced compared to the vehicle.

In addition, as can be seen in comparison in (B) and (C) of FIG. 3, the effects of naringenin and RA1 were compared, respectively, and both naringenin and RA1 reduced the size of the cerebral infarction site depending on a concentration, but RA1 (1×10⁻³ mg/kg) strongly reduced the size of the cerebral infarction site at a concentration 10,000 times lower than naringenin (10 mg/kg).

This shows that RA1 has very excellent efficacy in protective effect on brain cells even at a small concentration in the animal brain infarction model.

### Experimental Example 5

Effect of RA1 (VANL-100) on Cerebral Infarction Site while Administering RA1 (VANL-100) During Reperfusion Using Transient Middle Cerebral Artery Occlusion (t-MCAO) in Animal Experiments

First, in an animal model in which the middle cerebral artery of Sprague-Dawley rats (300 to 350 g; Charles River, Montreal, PQ, CAN) was closed for 30 minutes to create a cerebral infarction and then reperfused for 5 hours and 30 minutes (t-MCAO), RA1 (VANL-100) was administered at the beginning of reperfusion, after 1 hour of the beginning of reperfusion, and after 3 hours of the beginning of reperfusion, respectively, and compared with a case of administration of a vehicle.

As shown in FIG. 3(C), even at a very low concentration, the size of the cerebral infarction site was significantly reduced to about 47% to 70% compared to the case of the vehicle. It was observed that RA1 had a function of protecting brain cells for a considerable time before cerebral infarction or after performing cerebral infarction and reperfusion. This showed a possibility of further extending a time window of the treatment even after the onset of cerebral infarction.

As a result, as a result of using and examining an animal brain infarction model, among the novel compounds, RA1 had a protective effect on brain cells as a synergistic effect that is almost 10,000 times stronger at a very low concentration than that of a maternal component, naringenin or lipoic acid, and had an effect of significantly reducing the cerebral infarction site.

These effects represent that the novel substances had a potential as agents for prevention and treatment of cerebral nerve diseases such as stroke (cerebral infarction, and cerebral hemorrhage), Parkinson's disease, Alzheimer's disease, and cerebral nerve injury.

### Experimental Example 6

Measurement of Antioxidant Capacity and Inhibitory Effect of Tyrosinase for Novel Compounds as Skin External Preparations or Cosmetics

The antioxidant capacity of the novel compounds was measured by using a Blois method of measuring radical scavenging ability by DPPH which was widely known. A solution of DPPH by concentration was prepared in ethanol, and the initial absorbance was measured at 517 nm, and the absorbance over time was measured by adding a solution of the novel compound and the DPPH solution. The antioxidant capacity was expressed in % and an IC₅₀ value was obtained using a calculation program.

As shown in Table 1 below, most of the novel compounds showed stronger or somewhat weaker antioxidant capacity compared to naringenin and lipoic acid. In particular, the strong antioxidant capacity exhibited by RA1 showed the usefulness of this compound that may be applied to the skin.

The inhibitory effect on tyrosinase related to skin whitening and melanin formation was measured using the following experimental method. 10 μL of each sample solution at a different concentration and 20 μL of mushroom tyrosinase in a phosphate buffer (pH 6.5) were added to a 96-well microplate and added with 170 μL of a mixed solution in which a 1 mM L-tyrosine solution, a 50 mM potassium phosphate buffer (pH 6.5) and distilled water were contained in a ratio of 10:10:9. Samples dissolved in DMSO were diluted 30 times with distilled water and used. After incubation at 37° C. for 30 minutes, the absorbance of the reaction solution was measured at 490 nm using a GENios microplate reader (Tecan Austria GmbH, Austria). The inhibition degree of tyrosinase by the addition of the sample was expressed as a 50% inhibition rate (IC₅₀).

As shown in Table 1, the inhibitory effect of the novel compounds on the oxidation of L-trypsin promoted by tyrosinase usually had an IC₅₀ value of 32 to 350 μM, and some of the compounds had no inhibitory effect. In particular, RA1 and RA2 had IC₅₀ values of 32 μM and 58 μM, and had comparable or strong inhibitory effects compared to positive controls, kojic acid (44 μM) and arbutin (266 μM).

In conclusion, some of the novel compounds showed a possibility of being used as skin external preparations or cosmetics.

TABLE 1

| | Antioxidant | Inhibition of |
|---|---|---|
| Compound | capacity (%) | tyrosinase (μM) |
| Naringenin | 58.5 | 82 |
| Lipoic acid | 66.8 | 44 |
| Kojic acid | 28 | 44 |
| Arbutin | 36 | 266 |
| RA1 | 85.5 | 32 |
| RA2 | 77 | 58 |
| RA3 | 12.4 | 133 |
| RA4 | 22 | 260 |
| RA5 | 28.5 | 225 |
| RA6 | 14.6 | 350 |
| RA7 | — | — |
| RA8 | 11.5 | — |
| RA9 | 24 | — |

1. A compound represented by the following Chemical Formula 1: 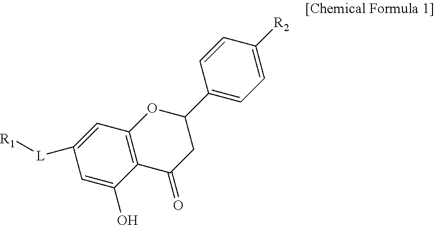
Wherein,
L is a direct bond, or a linker selected from —OCH₂CH₂—, —OCH₂CH₂OCH₂CH₂—, —OCH₂CH₂OCH₂CH₂OCH₂ CH₂—, —OCH₂CH₂OCH₂CH₂OCH₂CH₂OCH₂CH₂—, —OCH₂CH₂OCH₂CH₂OCH₂CH₂OCH₂CH₂OCH₂CH₂— and OCH₂CH₂OCH₂CH₂OCH₂CH₂OCH₂CH₂OCH₂CH₂OCH₂CH₂—, and
R₁ and R₂ are each independently OH or lipoic acid represented by the following Chemical Formula 2:

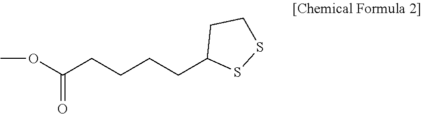

2. The compound of claim 1, wherein the compound of Chemical Formula 1 above includes the following compounds:

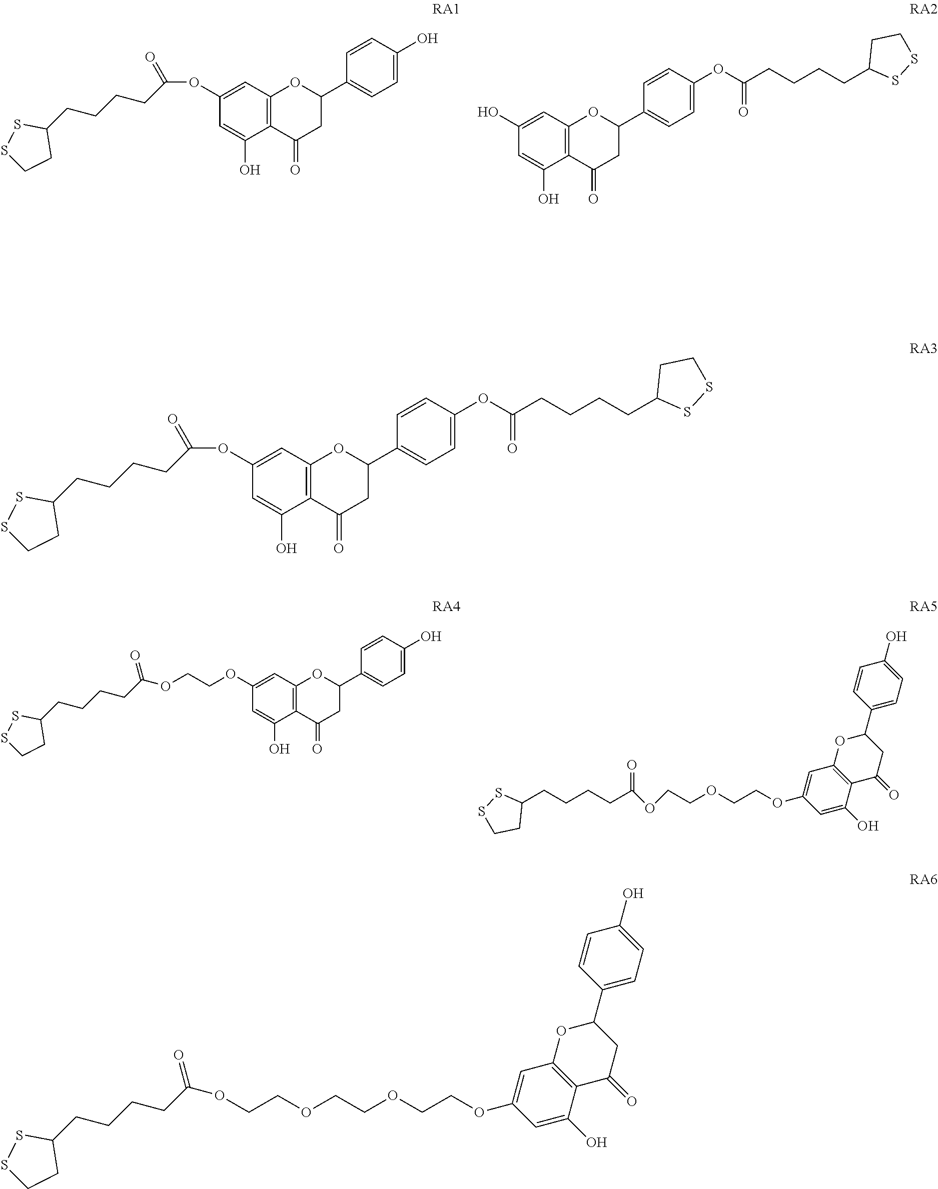 [SMILES: O=C(CCCCC1CCSS1)Oc1cc(O)c2c(c1)OC(c1ccc(O)cc1)CC2=O ; O=C(CCCCC1CCSS1)Oc1ccc(C2CC(=O)c3c(O)cc(O)cc3O2)cc1 ; O=C(CCCCC1CCSS1)Oc1ccc(C2CC(=O)c3c(O)cc(OC(=O)CCCCC4CCSS4)cc3O2)cc1 ; O=C(CCCCC1CCSS1)OCCOc1cc(O)c2c(c1)OC(c1ccc(O)cc1)CC2=O ; O=C(CCCCC1CCSS1)OCCOCCOc1cc(O)c2c(c1)OC(c1ccc(O)cc1)CC2=O ; O=C(CCCCC1CCSS1)OCCOCCOCCOc1cc(O)c2c(c1)OC(c1ccc(O)cc1)CC2=O]

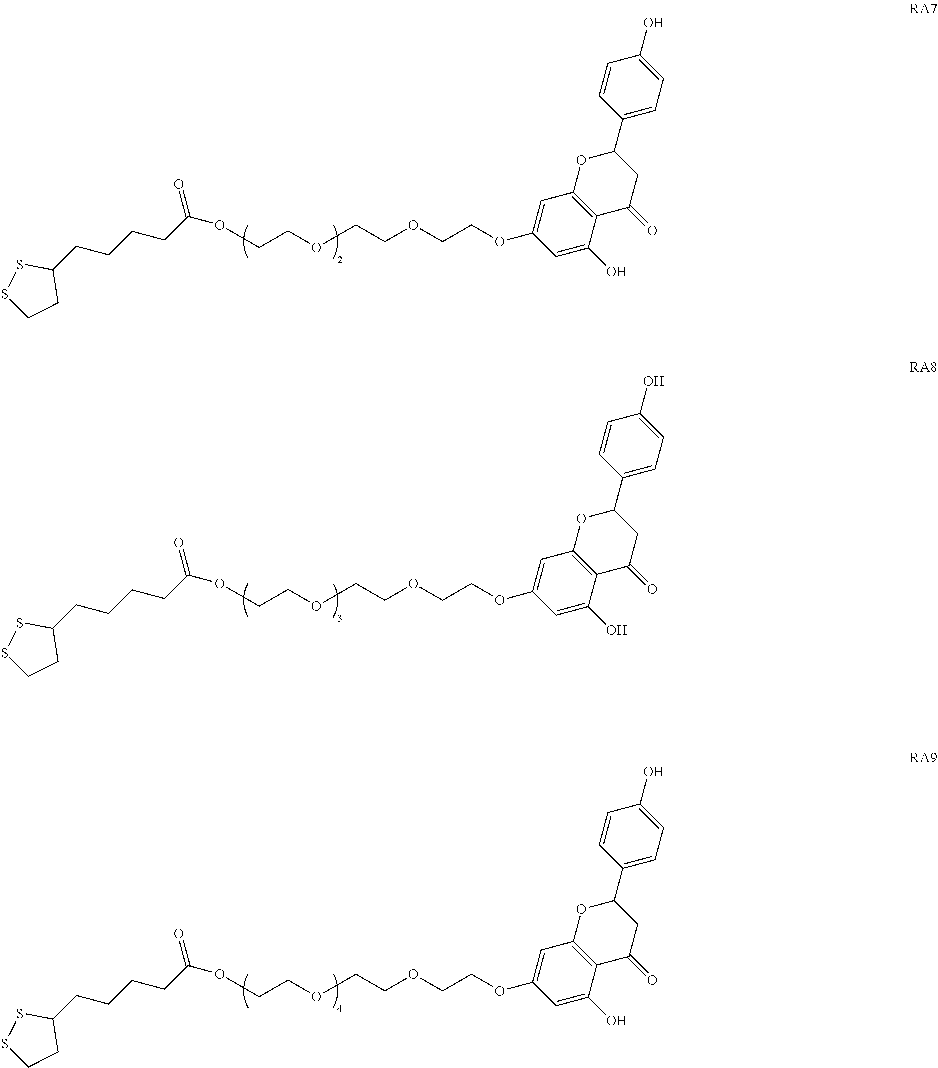

3. A pharmaceutical composition for treatment or prevention of stroke, cerebral infarction, cerebral hemorrhage, cognitive impairment, Parkinson's disease, Alzheimer's disease, vascular dementia, memory loss, and cerebral nerve injury, comprising the compound according to claim 1 or a pharmaceutical salt thereof as an active ingredient.

4. A functional health food for improvement or prevention of stroke, cerebral infarction, cerebral hemorrhage, cognitive impairment, Parkinson's disease, Alzheimer's disease, vascular dementia, memory loss, and cerebral nerve injury, comprising the compound according to claim 1 or a pharmaceutical salt thereof as an active ingredient.

5. A skin external preparation composition comprising the compound according to claim 1 or a pharmaceutical salt thereof as an active ingredient.

6. The skin external preparation composition of claim 5, wherein the skin external preparation composition has antioxidation, whitening, wrinkle relief, sun protection, acne, or anti-aging effects.

7. The skin external preparation composition of claim 5, wherein the skin external preparation composition is in the form of oil-in-water (O/W) or water-in-oil (W/O) including gels, packs, cream, essence, lotion, toner, and surfactants.

8. A method for preparing the compound according to claim 1, comprising reacting lipoic acid and naringenin.

9. A pharmaceutical composition for treatment or prevention of stroke, cerebral infarction, cerebral hemorrhage, cognitive impairment, Parkinson's disease, Alzheimer's disease, vascular dementia, memory loss, and cerebral nerve injury, comprising the compound according to claim 2 or a pharmaceutical salt thereof as an active ingredient.

10. A functional health food for improvement or prevention of stroke, cerebral infarction, cerebral hemorrhage, cognitive impairment, Parkinson's disease, Alzheimer's disease, vascular dementia, memory loss, and cerebral nerve injury, comprising the compound according to claim 2 or a pharmaceutical salt thereof as an active ingredient.

11. A skin external preparation composition comprising the compound according to claim 2 or a pharmaceutical salt thereof as an active ingredient.
